(19) United States
(12) Patent Application Publication
(10) Pub. No.: US 2006/0129022 A1
(43) Pub. Date: Jun. 15, 2006

(54) Method and apparatus for therapeutic treatment of inflammation and pain with low flux density, static electro-magnetic fields

(76) Inventors: Glenn E. Venza, Riverdale, NJ (US); Gerard Malady, Fort Lee, NJ (US); Robert Sylvester, River Edge, NJ (US)

Correspondence Address: JONES, TULLAR & COOPER, P.C., P.O. BOX 2266 EADS STATION, ARLINGTON, VA, 22202, US

(21) Appl. No.: 11/289,395

(22) Filed: Nov. 30, 2005

Related U.S. Application Data
(60) Provisional application No. 60/631,433, filed on Nov. 30, 2004.

(51) Int. Cl. A61N 1/00

(52) U.S. Cl. 600/13

(57) ABSTRACT

A portable Static Electro Magnetic Field (SEMF) generating device and method for the therapeutic treatment of inflammatory and painful disorders of the human limbs including fingers, hands, wrists, forearms, elbows, toes, feet and ankles preferably includes a wearable, portable battery powered solenoid or coil. The coil is configured to focus a substantially time invariant magnetic flux field onto a selected part of the body. The SEMF generating device's coil, when energized, works by slightly changing the charges associated with voltage-dependant ion channels in the cell membranes of the human body, thereby stabilizing abnormal fluid transport into the cells through aquaporin channels and the sodium ions relating to them, treating inflammation and altering the flow of calcium, chloride and potassium ions through their respective neuronal ion channels and treating pain.

This application is a continuation-in-part of and claims priority to the filing date of U.S. provisional application Ser. No. 60/631,433, filed Nov. 30^{th}, 2004, the entire disclosure of which is incorporated herein by reference.

### BACKGROUND OF THE INVENTION

1. Field of the Invention

The present invention relates to therapeutic application of electromagnetic fields to the body, and more particularly to a method and apparatus for therapeutic treatment of inflammation and pain by exposing an inflamed or painful area of the body to a low flux density static electro magnetic field (“SEMF”).

2. Discussion of the Prior Art

Many people suffer from musculoskeletal ailments and disorders of the limbs and experience inflammation and pain. These disorders include Carpal Tunnel Syndrome (Median Nerve Entrapment), Arthritic Wrist Pain, Lateral Epicondylitis, Medial Epicondylitis, DeQuervain's Disease, Forearm Tendonitis, Wrist Tendonitis, Trigger Finger, Intersection Syndrome, Hand-Arm Vibration Syndrome, Ulnar Nerve Entrapment, Acute Ankle Sprains, Posterior Tibial Tendonitis, Achilles Tendonitis, Plantar Fascitis and Morton's Neuroma.

In response, many have turned to use of over the counter pain medications or sought a variety of other remedies that have proven to be inconvenient, ineffective, expensive or painful. Some have experimented with electric fields or with magnetic fields.

The planet Earth has a static, surface geomagnetic field that is superficially similar to that of a permanent bar magnet. The coordinated motion of negatively charged electrons within (e.g., iron) atoms creates the magnetic fields in permanent magnets. Those coordinated orbits become randomized at the Curie point temperature of 1043 degrees Kelvin and magnetism degrades. The temperature of the Earth's core however, is much higher than that and yet the Earth's geomagnetic field remains. One can surmise therefore that the Earth's geomagnetic field and its related magnetosphere are not due to magnetized iron deposits. The latest school of thought is that as portions of the molten outer core of the Earth cool and fall inward, oceans of iron rich liquid magma rise towards the surface. The rotation of the planet forces the magma into a helical motion, regenerating the Earth's magnetism.

For centuries, traditional Chinese medicine has used lodestones or magnets to treat diseases and pain and to encourage healing. Application of this type of therapy was used in conjunction with the map of acupuncture points of the human body. The lodestone was placed at or near an acupuncture point relating to the complainant's described problem and, hopefully, the sought after relief followed. The Static Magnetic Field (SMF) applied to an acupuncture point either alone or together with an acupuncture needle was a safe and effective therapy. This permanent magnet therapy has become a booming industry in recent years. Magnetic gloves, socks, insoles, straps, mattress pads, belts, braces, jewelry etc. are available through print ads, the Internet, infomercials and the shelves of local pharmacies with worldwide sales in excess of five billion dollars. Until recently, the therapeutic action of SMFs was a complete mystery, however studies within the last decade have shed new light on how static magnetic fields influence cells and help the body to heal itself.

The safety of SMFs has led to a useful diagnostic tool, namely Magnetic Resonance Imaging (MRI) as described in U.S. Pat. No. 3,024,410. The MRI scanner is a tube surrounded by a very large circular magnet. The magnet creates a strong magnetic field, often as much as 70,000 times stronger than the geomagnetic field, and yet is harmless to the patient. This magnetic field aligns the protons of hydrogen atoms in the body, allowing a computer to process the information and create an image.

At the other end of the magnetic picture, the dangers and risks of high frequency magnetic radiation are well known and well documented. Three or four decades ago scientists worldwide began investigations into the safety of Electro Magnetic Fields (EMFs) generated by high-tension power lines. As current travels through a conductor a magnetic field is generated perpendicularly to the direction of the flow of the current.

Supposed cancer clusters allegedly caused by these low frequency EMFs precipitated these investigations and numerous studies of the subject. These 60 cycle/second EMFs were an enigma until these studies showed that there was not sufficient evidence to link cancer clusters and the magnetic fields. The studies that were launched by this investigation all dealt with frequency modulated magnetic fields. In other words, the field was turned on and off (pulsed) any specified times per second using a variety of wave geometries such as square waves, saw tooth waves (triangular) and sine waves. These myriad studies varied the frequency of the field from just a few cycles per second to many thousands of cycles per second. Many of the scientists involved in these studies knew of the subjective successes that experimenters had with SMFs and thus studied the efficacy of Pulsed Electro Magnetic Fields (PEMFs) on human ailments ranging from simple headaches to fibromyalgia to the treatment of cancer, with results ranging from promising to effective.

For years, science has recognized that the roughly sixty-five trillion cells in the human body generate, employ and exude electric and magnetic charges. For example Electroencephalograms (EEGs) U.S. Pat. No. 2,409,033 and Electrocardiograms (EKGs) U.S. Pat. No. 2,262,936 are the electrical measurements of brain and heart functions respectively. There are current studies being done to evaluate the effectiveness of Magneto-Encephalography (MEG) U.S. Pat. No. 4,913,152, which is used to isolate areas of the brain causing epilepsy and Magneto-Cardiography (MKG) U.S. Pat. No. 5,594,849, to evaluate cardiac function. As PEMFs and SEMFs become state of the art for treatment of painful disorders a new device called a SQUID (Super-conducting QUantum Interference Device) U.S. Pat. No. 4,591,787, will be used to measure the minute fields, both electrical and magnetic, that make up the human body and how these externally applied fields such as PEMFs and SEMFs interact with and affect the body's internally generated electrical and magnetic fields. There is one major difference between electric and magnetic charges as they relate to the human body. In order to either measure (EKG, EEG) an electrical charge generated by the body, or to deliver (DEFIBRILLATOR U.S. Pat. No. 3,050,695, TENS [Transcutaneous Electrical Nerve Stimulator U.S. Pat. No. 2,375,575]) an electrical charge to the body, physical contact of conducting electrodes must be used. This is not true of a magnetic field, as we know by the use of an MRI. Not only is the magnetic field effective without contact, but it is also barely affected by hard tissue such as bone, allowing penetration of the field to all soft tissue for diagnosis.

Appropriate magnetic fields have not been exploited as a mechanism for pain relief in a practical form, however, for use by individuals when engaged in their everyday activities.

There is a need, therefore, for a safe and effective portable device for treating musculoskeletal ailments and disorders of the limbs by reducing inflammation and pain.

### SUMMARY OF THE INVENTION

The present invention includes a method and apparatus for treating physical disorders of the human body using a Low Flux Density, Static Electro-Magnetic Field (SEMF). The invention preferably comprises a safe and effective portable device which the patient can operate. The non-heating, non-ionizing SEMF generated by the method and apparatus of the present invention effectively treats a wide variety of musculoskeletal ailments and disorders of the limbs by reducing inflammation and pain. These disorders include but are not limited to Carpal Tunnel Syndrome (Median Nerve Entrapment), Arthritic Wrist Pain, Lateral Epicondylitis, Medial Epicondylitis, DeQuervain's Disease, Forearm Tendonitis, Wrist Tendonitis, Trigger Finger, Intersection Syndrome, Hand-Arm Vibration Syndrome, Ulnar Nerve Entrapment, Acute Ankle Sprains, Posterior Tibial Tendonitis, Achilles Tendonitis, Plantar Fascitis and Morton's Neuroma.

A portable Static Electro Magnetic Field (SEMF) generating device and method for the therapeutic treatment of inflammatory and painful disorders is configured to be held against or supported upon human limbs including fingers, hands, wrists, forearms, elbows, toes, feet or ankles and preferably includes a wearable, portable, battery powered solenoid or coil.

The coil is configured to aim or focus a substantially time invariant magnetic flux field onto and through a selected part of the body. The SEMF generating device's coil, when energized, works by slightly changing the charges associated with voltage-dependant ion channels in the cell membranes of the selected part of the body, thereby stabilizing abnormal fluid transport into the cells through aquaporin channels and the sodium ions relating to them, treating inflammation and altering the flow of calcium, chloride and potassium ions through their respective neuronal ion channels and treating pain.

The above and still further features and advantages of the present invention will become apparent upon consideration of the following detailed description of a specific embodiment thereof, particularly when taken in conjunction with the accompanying drawings, wherein like reference numerals in the various figures are utilized to designate like components.

### BRIEF DESCRIPTION OF THE DRAWINGS

FIG. 1 is a top or plan view, in elevation, of a wearable static electro magnetic field generating therapeutic device, illustrating the position of the indicating LED and the actuating switch, in accordance with the present invention.

FIG. 2 is a side view in elevation of the wearable static electro magnetic field generating therapeutic device of FIG. 1, illustrating the contoured housing and the strap or affixing unit and showing, in hidden lines, location and orientation of the field generating solenoid coil within the housing unit, in accordance with the present invention.

FIG. 3 is a diagram showing, in perspective, the placement and orientation of the operating electrical and magnetic field generating components of the wearable static electro magnetic field generating therapeutic device of FIGS. 1 and 2, in accordance with the present invention.

FIG. 4 is a schematic diagram showing the electrical connections among the operating electrical and magnetic field generating components of the wearable static electro magnetic field generating therapeutic device of FIGS. 1-3, in accordance with the present invention.

FIG. 5 is a diagram illustrating the method for applying the wearable static electro magnetic field generating therapeutic device of FIGS. 1-4, and illustrating an exemplary fitment appropriate for treatment of Lateral Epicondylitis (Tennis Elbow) on the anterior, human right arm, in accordance with the present invention.

FIG. 6 is a diagram illustrating the method for applying the wearable static electro magnetic field generating therapeutic device of FIGS. 1-4, and illustrating an exemplary fitment appropriate for treatment of Medial Epicondylitis (Golfer's Elbow) on the anterior, human right arm, in accordance with the present invention.

FIG. 7 is a diagram illustrating the method for applying the wearable static electro magnetic field generating therapeutic device of FIGS. 1-4, and illustrating an exemplary fitment appropriate for treatment of Ulnar Nerve Entrapment (Cubital Tunnel Syndrome) on the anterior, human right arm, in accordance with the present invention.

FIG. 8 is a diagram illustrating the method for applying the wearable static electro magnetic field generating therapeutic device of FIGS. 1-4, and illustrating an exemplary fitment appropriate for treatment of Forearm Tendonitis on the posterior, human right arm, in accordance with the present invention.

FIG. 9 is a diagram illustrating the method for applying the wearable static electro magnetic field generating therapeutic device of FIGS. 1-4, and illustrating an exemplary fitment appropriate for treatment of DeQuervain's Disease (Blackberry Thumb) on the posterior, human right hand, in accordance with the present invention.

FIG. 10 is a diagram illustrating the method for applying the wearable static electro magnetic field generating therapeutic device of FIGS. 1-4, and illustrating an exemplary fitment appropriate for treatment of Intersection Syndrome on the posterior, human right hand, in accordance with the present invention.

FIG. 11 is a diagram illustrating the method for applying the wearable static electro magnetic field generating therapeutic device of FIGS. 1-4, and illustrating an exemplary fitment appropriate for treatment of Trigger Finger on the posterior, human right hand, in accordance with the present invention.

FIG. 12 is a diagram illustrating the method for applying the wearable static electro magnetic field generating therapeutic device of FIGS. 1-4, and illustrating an exemplary fitment appropriate for treatment of Wrist Tendonitis on the anterior, human right hand, in accordance with the present invention.

FIG. 13 is a diagram illustrating the method for applying the wearable static electro magnetic field generating therapeutic device of FIGS. 1-4, and illustrating an exemplary fitment appropriate for treatment of Hand-Arm Vibration Syndrome (HAVS) on the anterior, human right hand, in accordance with the present invention.

FIG. 14 is a diagram illustrating the method for applying the wearable static electro magnetic field generating therapeutic device of FIGS. 1-4, and illustrating an exemplary anterior fitment appropriate for treatment of Median Nerve Entrapment (Carpal Tunnel Syndrome) on the anterior, human right hand, in accordance with the present invention.

FIG. 15 is a diagram illustrating the method for applying the wearable static electro magnetic field generating therapeutic device of FIGS. 1-4, and illustrating an exemplary posterior fitment appropriate for treatment of Median Nerve Entrapment (Carpal Tunnel Syndrome) on the posterior of the human right hand, in accordance with the present invention.

FIG. 16 is a diagram illustrating the method for applying the wearable static electro magnetic field generating therapeutic device of FIGS. 1-4, and illustrating an exemplary fitment appropriate for treatment of Acute Ankle Sprains on the dorsal, human right foot, in accordance with the present invention.

FIG. 17 is a diagram illustrating the method for applying the wearable static electro magnetic field generating therapeutic device of FIGS. 1-4, and illustrating an exemplary fitment appropriate for treatment of Morton's Neuroma on the dorsal, human right foot, in accordance with the present invention.

FIG. 18 is a diagram illustrating the method for applying the wearable static electro magnetic field generating therapeutic device of FIGS. 1-4, and illustrating an exemplary fitment appropriate for treatment of Plantar Fascitis on the plantar, human right foot, in accordance with the present invention.

FIG. 19 is a diagram illustrating the method for applying the wearable static electro magnetic field generating therapeutic device of FIGS. 1-4, and illustrating an exemplary fitment appropriate for treatment of Achilles Tendonitis on the posterior, human right ankle, in accordance with the present invention.

FIG. 20 is a diagram illustrating the method for applying the wearable static electro magnetic field generating therapeutic device of FIGS. 1-4, and illustrating an exemplary fitment appropriate for treatment of Posterior Tibial Tendonitis on the posterior, human right ankle, in accordance with the present invention.

FIG. 21 is a diagram illustrating the Synaptic Junction within the human body; in accordance with the method of the present invention, application of the SEMF causes calcium ion channels to open, and as the action potential reaches the synaptic cleft, the inrush of ions triggers neurotransmitter vesicles to release their contents of GABA-A and GABA-B 55; these neurotransmitters, in conjunction with the SEMF, cause potassium ion channels and chloride ion channels to open, forcing the post synaptic neuron to maintain its resting potential.

FIG. 22 is a diagram illustrating the magnetic field produced by a single current loop, where “I” indicates the applied current and direction thereof and “B” indicates the electro magnetic field with arrows showing South Pole to North Pole radiation.

FIG. 23 is a view of the magnetic field of a solenoid coil from Ampere's Law, where I is the applied current and the magnetic field, indicated by lines and arrows, is bipolar in nature.

FIG. 24 is a diagram illustrating the right hand rule where “I” indicates the applied current with the thumb pointing in the direction of that current and B is the generated magnetic field with the fingers indicating the direction of magnetic flux from South Pole to North Pole.

### DETAILED DESCRIPTION OF THE INVENTION

Before turning to a detailed description of the preferred embodiment, the theoretical underpinnings of the present invention will be set forth. Static Electro Magnetic Fields (SEMFs) produced in the fashion that this invention generates them, and their ability to effectuate the reduction of pain and inflammation are based on a plethora of electric, magnetic and biological laws and theories.

MICHAEL FARADAY had observed how iron filings form patterns of lines under the influence of a magnet. He concluded that space is filled with these invisible lines, forming what we now call a field. He used his idea to explain in 1845 why some substances are diamagnetic (developing a magnetic field opposite to the one surrounding them) and others paramagnetic (developing a magnetic field parallel to the one surrounding them). When Faraday discovered that a magnetic field could affect the polarization of light, he proposed in 1845 that light might be waves in the lines of force of electromagnetism.

ALBERT EINSTEIN showed, using special relativity, that electric and magnetic fields are two aspects of the same thing (a rank-2 tensor), and that one observer may perceive a magnetic force where a moving observer perceives only an electrostatic force. Thus, using special relativity, magnetic forces are a manifestation of electrostatic forces of charges in motion and may be predicted from knowledge of the electrostatic forces and the movement (relative to some observer) of the charges.

Electric currents produce MAGNETIC FIELDS, which can be macroscopic currents in wires, or microscopic currents associated with electrons in atomic orbits. Magnetic field sources are essentially dipolar in nature, having a north and south magnetic pole. In physics, a magnetic field is an entity produced by moving electric charges or electric currents that exerts a force on other moving charges.

GAUSS' LAW FOR MAGNETISM states that for a magnetic dipole, any closed surface the magnetic flux directed inward toward the South Pole will equal the flux outward from the North Pole. The net flux will always be zero for dipole sources.

AMPERE'S LAW states the magnetic field in space around an electric current is proportional to the electric current which serves as its source, and the BIOT-SAVART LAW relates magnetic fields to the currents which are their sources. Finding the magnetic field resulting from a current distribution involves the vector product and is inherently a calculus problem. The direction of the magnetic field contribution follows the right hand rule illustrated for a straight wire. This direction arises from the vector product nature of the dependence upon electric current.

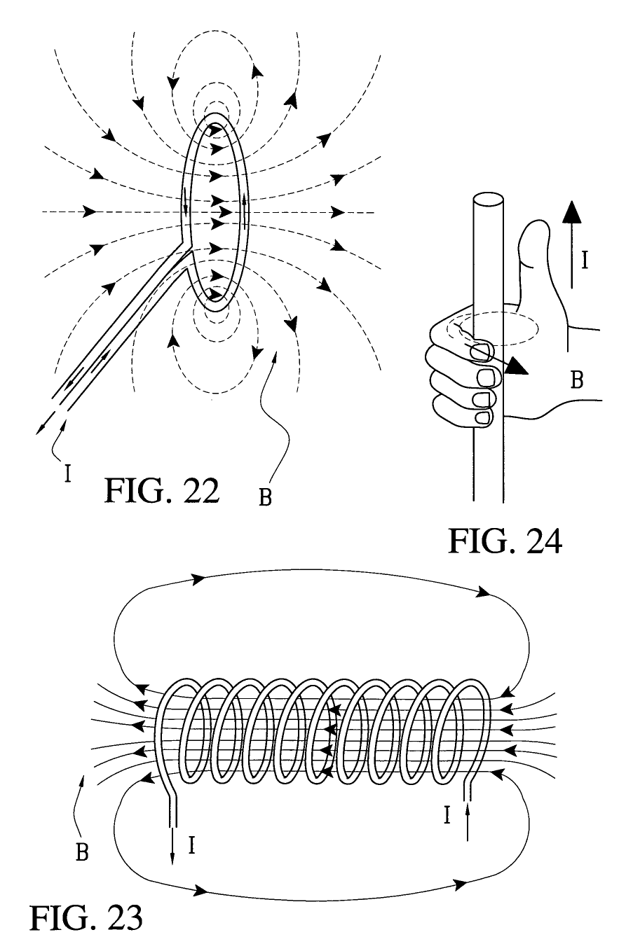

The RIGHT HAND RULE is a useful mnemonic for visualizing the direction of a magnetic force. As best seen in FIG. 24, the thumb of the right hand points in the direction of the current flow in a conductor and the fingers wrap in the direction of the magnetic field. As best seen in FIGS. 22 and 23, the flux lines illustrating the magnetic field in a current loop or (or coil turn) are more concentrated in the center of the loop than outside the loop. Examining the direction of the magnetic field produced by a current-carrying segment of wire shows that all parts of the loop contribute magnetic field in the same direction inside the loop. Stacking multiple loops (as shown in FIG. 23) concentrates the field even more into what is called a solenoid.

A SOLENOID is a coil of wire that can be used to generate a nearly uniform magnetic field. Such coils have an enormous number of practical applications as in this invention, the reduction of inflammation and pain.

The term ELECTROMAGNETISM comes from the fact that the electric and magnetic fields are closely intertwined, and, under many circumstances, it is impossible to consider the two separately. For instance, a changing magnetic field gives rise to an electric field; this is the phenomenon of electromagnetic induction. The force that the electromagnetic field exerts on electrically charged particles, called the electromagnetic force, is one of the four fundamental forces. The other fundamental forces are the strong nuclear force (which holds atomic nuclei together), the weak nuclear force (which causes certain forms of radioactive decay), and the gravitational force. All other forces are ultimately derived from these fundamental forces. However, it turns out that the electromagnetic force is the one responsible for practically all the phenomena one encounters in daily life, with the exception of gravity. Roughly speaking, all the forces involved in interactions between atoms can be traced to the electromagnetic force acting on the electrically charges protons and electrons inside the atom. It also includes all forms of chemical phenomena, which arise from interactions between electron orbitals.

AQUAPORIN and VOLTAGE-DEPENDENT ION CHANNELS, the shared subjects of the 2003 Nobel Prize in Chemistry, are central to the function of nerves and muscles in the human body. Voltage-sensing “paddles” act as gates for the channels through the protein membrane of each cell. When the outside of the membrane becomes negatively charged, the positively charged paddles are attracted outward. This action opens the channels allowing ionic flow. Calcium, sodium, chloride and potassium channels are all related by being voltage dependent and having the same voltage sensor in their process. The ubiquity of aquaporin and ion channels throughout the body will allow this invention to be effective for virtually all inflammation and pain.

A static electromagnetic field (“SEMF”) applied to the body in a therapeutic fashion alters the electrical charge of the cell membrane, cytoplasm and surrounding fluid, causing some metabolic operations to accelerate, such as vasodilation, edema reduction, and calcium, potassium and chloride ion motility; while causing other metabolic operations to retard, such as nerve cell firing and inflammation. As SEMFs gain popularity with the suffering masses, the biological affects these fields have on the living human body are being studied, although the “mechanism of action” of SEMFs is still somewhat of an enigma. Much more is known and accepted regarding SEMF safety and efficacy than the “mechanism of action” and safety of many pharmaceuticals.

As previously stated, the only familial link that the SEMF has with the permanent magnet is the inherent safety of the field. SEMF therapy is much more akin to PEMF therapy, but unlike a PEMF, a SEMF is a non-heating, non-ionizing field. However, the efficacy of PEMFs, and by association SEMFs, cannot be overlooked (not only in the effective regulation of pain, but even chronic nonunion fractures have been successfully treated).

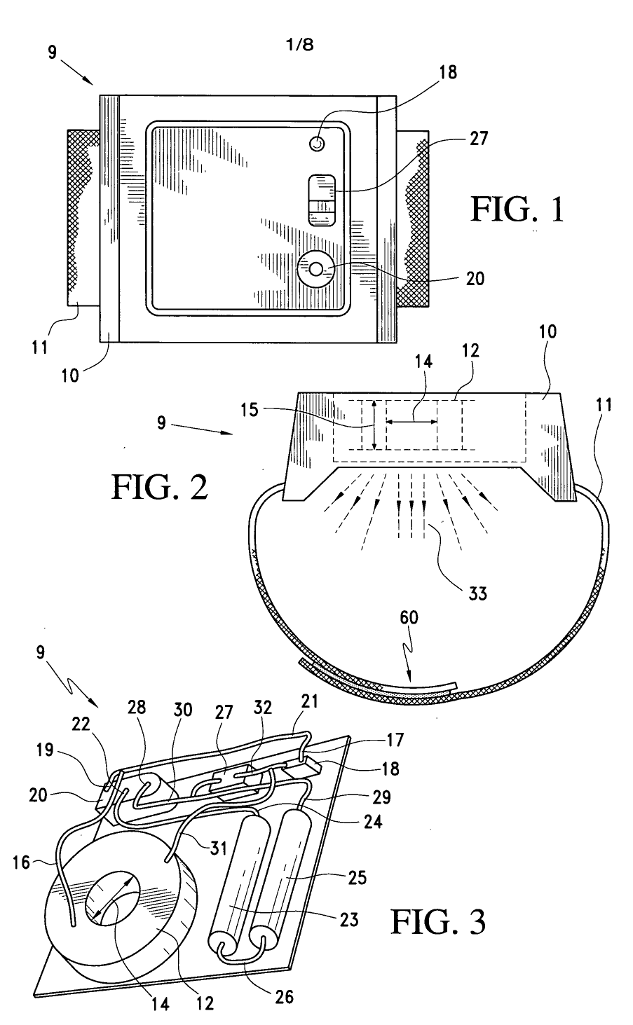

Turning now to a more detailed description of the embodiments of FIGS. 1-21, wearable static electro magnetic field generating therapeutic device 9, through the coupling of a non-oscillating DC voltage supply and a non-ferrous solenoid coil 12, generates a Static Electro Magnetic Field (SEMF) with a magnitude of approximately twenty five Gauss at a distance of zero cm, gradually falling off to a level that cannot be distinguished from the geomagnetic field at 17.5 cm. The non-oscillating DC voltage supply, providing about a 2.66 Volt potential and a current of approximately 95 milliamps, allows the invention to operate at 0 cycles-per-second (Hertz) while delivering a magnetic field strength of about −0.0025 Tesla (A/m), an electric field strength of about 0.938708 V/m and a power density of about 0.0023374 W/m². The SEMF that is emitted is a gradient field increasing from the outer circumference of the geometry of the solenoid coil 12 to maximum field strength at the planar center, as measured proximate the contoured lower wall of housing 10.

The instant applicants have posited that the magnetic and electric properties of the generated field effectuate minute voltage variances on cell membranes and on surrounding fluids. FIG. 21 illustrates the Synaptic Junction within the human body. In accordance with the method of the present invention, application of the SEMF causes calcium ion channels 50 to open. As the action potential reaches the synaptic cleft 54, the inrush of ions triggers neurotransmitter vesicles 53 to release their contents of GABA-A and GABA-B 55. These neurotransmitters, in conjunction with the SEMF, cause potassium ion channels 51 and chloride ion channels 52 to open, forcing the post synaptic neuron 56 to maintain its resting potential. As best seen in FIG. 21, the generated negatively charged field, by electromagnetic induction, causes a permutation of the inherent voltage of the cell membrane. This deviation from a typical voltage causes voltage-sensing “paddles” in the aquaporin channels of the cell membrane to open, enhancing the flow of cellular and intracellular water and related sodium ions. Water molecules, at the endocellular level, cluster together into the three-dimensional shape of the dodecahedron around localized sodium ions. These in turn, aggregate into very large clusters within icosahedron shaped, magnetically attracted fascine, which are much too large to traverse the aquaporin channels of the cell membrane. The application of the SEMF of this invention disrupts the magnetic attraction of the icosahedra clusters, allowing the dodecahedral bundles to separate and pass through the cell membrane. This twofold action on hydrated sodium and aquaporin channels at the sub-cellular level results in a reduction of localized edema.

It is also posited that magnetically induced ionic transmembrane conduction is a result of this invention. When the SEMF of the invention is introduced to a region of the body experiencing pain, it causes calcium ion channels to open allowing an inward rush of calcium ions as the action potential reaches the synaptic cleft. This triggers neurotransmitter vesicles to release their contents into the synaptic cleft (exocytosis). The neurotransmitters most likely released under the influence of a SEMF are Gamma Amino Butyric Acid-A and B (GABA-A and GABA-B) which inhibit nerve impulse transmission. The SEMF, working in conjunction with the neurotransmitters GABA-A and GABA-B, cause the “paddles” in potassium ion channels of the receptor cell membrane to open to sanction the outward flow of potassium ions as well as prompting chloride ion channel “paddles” to open allowing a cellular influx of chloride ions forcing the post-synaptic neuron to maintain its resting membrane potential of −70 mV affecting a reduction of acute as well as chronic pain.

Referring to FIGS. 1-4, the preferred embodiment of the invention includes a non-ferrous, non-conductive housing unit 10 made, preferably from a tough, resilient plastic material, and has a top or upper surface including an electrical connector 20 or charging socket adapted to receive a detachable connector for recharging an internally carried battery power supply. The upper surface of housing 10 also includes a visible indicator of operating state such as a green Light Emitting Diode (LED) 18, and provides an access slot adapted to permit access to a user actuable enabling switch such as single-pole, single-throw (SPST) switch 27. The bottom or lower surface of housing 10 is configured to make contact with the body when worn, and so, in the illustrative embodiment, is contoured to provide a substantially planar back contact surface bounded by opposing angled wall segments configured to position the coil 12 in close proximity to an area of the body to be treated, as best seen in FIG. 2.

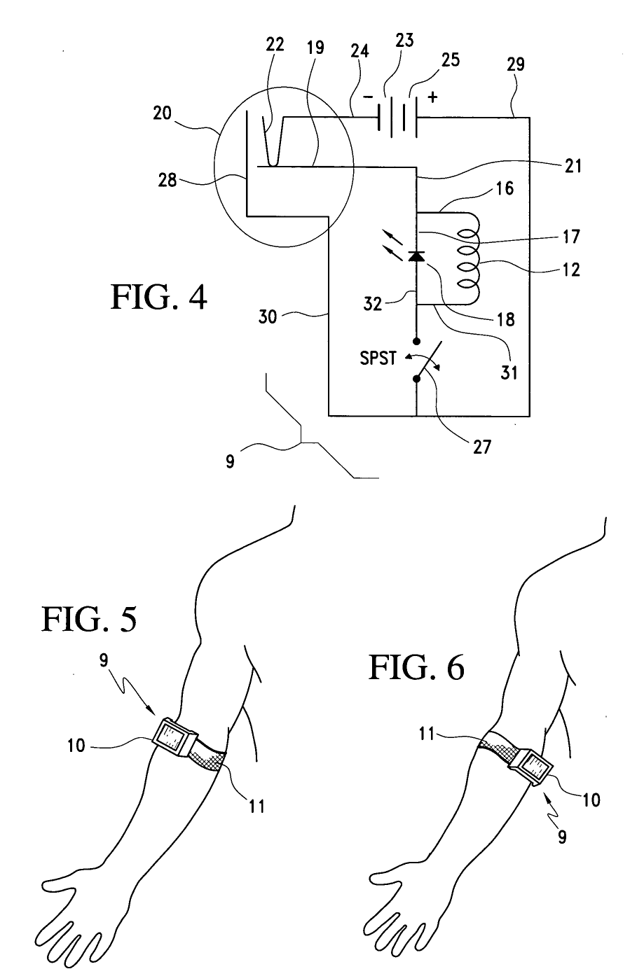

Housing 10 is supported by and carries a releasable flexible strap or affixing unit 11. As illustrated by the schematic in FIG. 4 the Static Electro Magnetic Field generating unit 9 is carried within and protected by housing 10 and can be attached to the body in many possible orientations, as will be discussed in greater detail, below.

Referring to the component layout diagram of FIG. 3 and schematic diagram of FIG. 4, Therapeutic treatment coil or solenoid 12 comprises a plurality of turns of conductor (e.g., copper wire) wound onto a substantially cylindrical non-ferrous bobbin, former or form 13. Coil form 13, due to its inherent geometry causes the solenoid coil 12 to have a fixed internal diameter 14 of about three quarters of an inch and a fixed height 15 of about one half inch. Coil 12 is terminated in a first (or start) lead 16 and a second (or finish) lead 31. Start lead 16 of solenoid coil 12 is permanently affixed and conductively bonded to the cathode 17 of, preferably, a green LED 18, typically a Panasonic p/n LN322GP, by a soldering process. This connection is also permanently affixed to the normally closed (N-C) pole 19 of an electrical connector for battery charging such as the female charging socket 20 by a length of insulated copper wire 21 and is soldered thereto. The second N-C negative pole 22 of the female charging socket 20 is permanently affixed to the negative pole of the first battery 23, preferably rechargeable (e.g., NiMH AAA) battery, by a length of insulated copper wire 24 and is soldered thereto. The positive pole of the first rechargeable battery 23 is soldered to the negative pole of a second rechargeable battery 25 using solderable conductive tabs 26 contacting the batteries 23 and 25. The positive pole of the second battery 25 is permanently affixed to one pole of the SPST switch 27, typically a Cannon-ITT p/n GS02MCKE, and the positive pole 28 of the female charging socket 20 is connected by insulated copper wires 29 and 30 and soldered thereto. The finish lead 31 of the solenoid coil 12 is preferably permanently connected to the anode 32 of the LED 18 and the other pole of the SPST switch 27 by a soldering process.

When switch 27 is in a closed condition, it closes the circuit causing current to flow at an amplitude of about ninety five milliamps (for a supply voltage 2.66 volts DC) through the field generating unit 9. The green LED 18 is energized to provide an “ON” indicator and the solenoid coil 12 generates a Static Electro Magnetic Field 33 of about 25 Gauss. When the batteries 23 and 25 are depleted and can no longer provide adequate current, the user may then employ an external charging device (not shown) including a standard pin connector adapted to be plugged into the female charging socket 20. Standard “wall wart” DC rechargers are available for use with any mains supply (e.g., 120 Volt in the US) wall outlet.

When a male charging plug is inserted into the female charging socket 20, the solenoid coil 12, the SPST switch 27 and the green LED 18 are all physically disconnected or removed from the circuit by the N-C negative poles 19 and 22 being forced into an open condition, safely allowing the portable power supply (e.g., batteries 23 and 25) to be recharged. This opening of the N-C negative poles 19 and 22 renders the embodiment of the field generating unit 9 useless while charging, creating a safe condition.

The housing unit 10 is preferably a molded thermoplastic case, and the mold defines the vias or ports for the LED 18, the female charging socket 20 and the SPST switch 27; as well as internal structural protrusions to support and stabilize the batteries 23 and 25 and maintain placement of the solenoid coil 12.

The strap or affixing unit 11 is a preferably a segment of flexible resilient web material terminated at first and second ends with sections of affixed hook and loop type fasteners (e.g., at 60 in FIG. 2), or can be configured using the same structural features of a watchband or other band, sized to fit on parts of the body, as shown in FIGS. 5-20. Alternatively, strap or affixing unit 11 may be an elastic or stretchable band dimensioned to closely fit a selected appendage.

In use, the wearable static electro magnetic field generating therapeutic device 9 of FIGS. 1-4, is fitted to an affected part of the body and switch 27 is actuated, whereby solenoid coil 12 is energized to provide a therapeutic effect on a selected appendage or joint within the human body. Referring to FIG. 5, an exemplary fitment appropriate for treatment of Lateral Epicondylitis (Tennis Elbow) on the anterior, human right arm, is illustrated. The user dons or wears strap 11 and housing 10 in an orientation placing housing 10 against the anterior, human right arm, and then actuates or energizes solenoid coil 12 to focus magnetic flux into and through the elbow region's Lateral Epicondylitis.

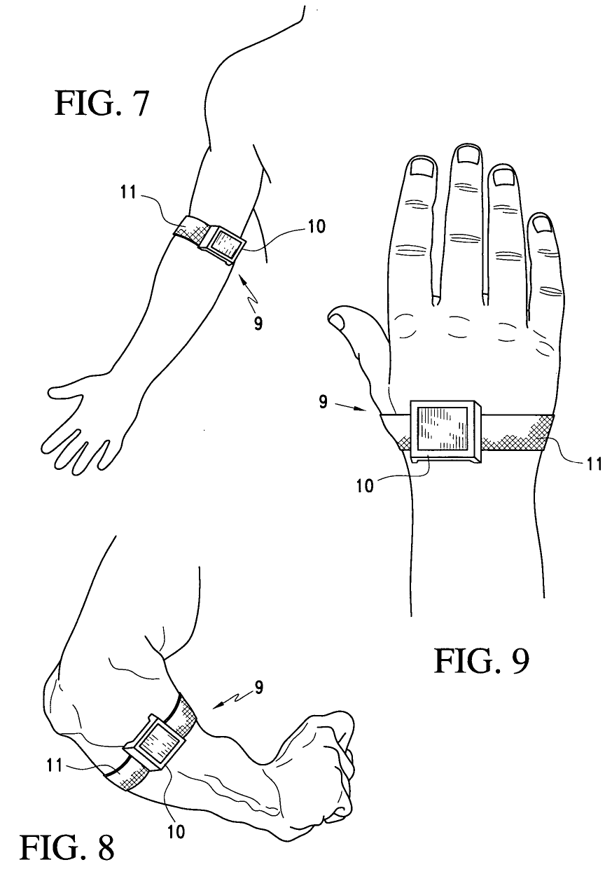

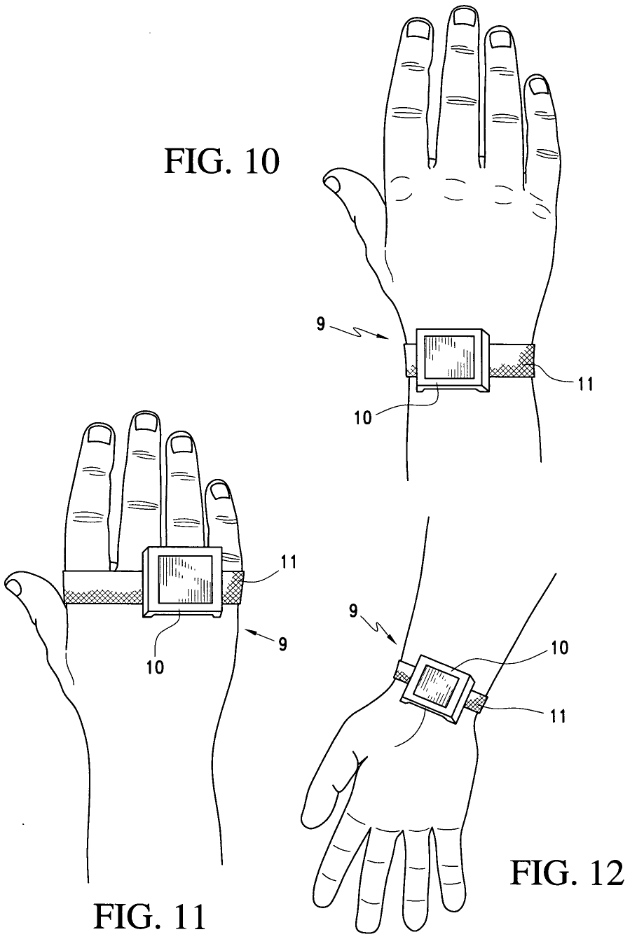

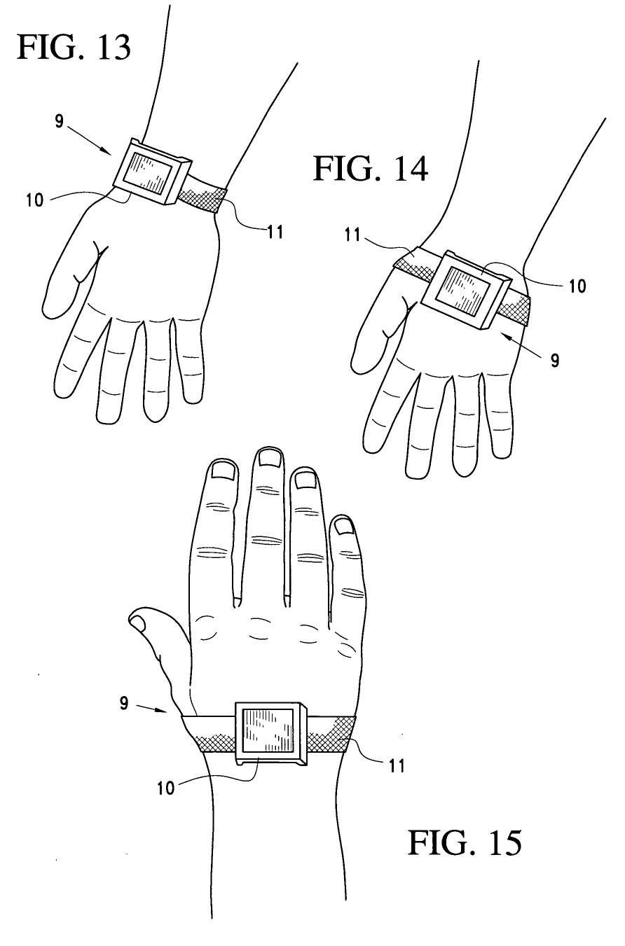

The method is adapted to many other treatments; for example, FIG. 6 is a diagram illustrating the method for applying the wearable static electro magnetic field generating therapeutic device 9, and illustrating an exemplary fitment appropriate for treatment of Medial Epicondylitis (Golfer's Elbow) on the anterior, human right arm, in accordance with the present invention. FIG. 7 illustrates the method for applying the wearable static electro magnetic field generating therapeutic device 9, showing an exemplary fitment appropriate for treatment of Ulnar Nerve Entrapment (Cubital Tunnel Syndrome) on the anterior, human right arm, and FIG. 8 illustrates the method for applying the wearable static electro magnetic field generating therapeutic device 9 in an exemplary fitment appropriate for treatment of Forearm Tendonitis on the posterior of a human right arm. FIG. 9 is a diagram illustrating the method for applying the wearable static electro magnetic field generating therapeutic 9 and showing an exemplary fitment appropriate for treatment of DeQuervain's Disease (Blackberry Thumb) on the posterior of the human right hand, and FIG. 10 shows the method for applying wearable static electro magnetic field generating therapeutic device 9 in an exemplary fitment appropriate for treatment of Intersection Syndrome on the posterior, human right hand. FIG. 11 shows the method for applying static electro magnetic field generating therapeutic device 9 in an exemplary fitment appropriate for treatment of Trigger Finger on the posterior, human right hand, and FIG. 12 shows the method for applying the wearable static electro magnetic field generating therapeutic device 9 in an exemplary fitment appropriate for treatment of Wrist Tendonitis on the anterior, human right hand. FIG. 13 illustrates the method for applying SEMF generating therapeutic device 9 of FIGS. 1-4, and shows an exemplary fitment appropriate for treatment of Hand-Arm Vibration Syndrome (HAVS) on the anterior, human right hand, while FIG. 14 shows an exemplary anterior fitment appropriate for treatment of Median Nerve Entrapment (Carpal Tunnel Syndrome) on the anterior, human right hand.

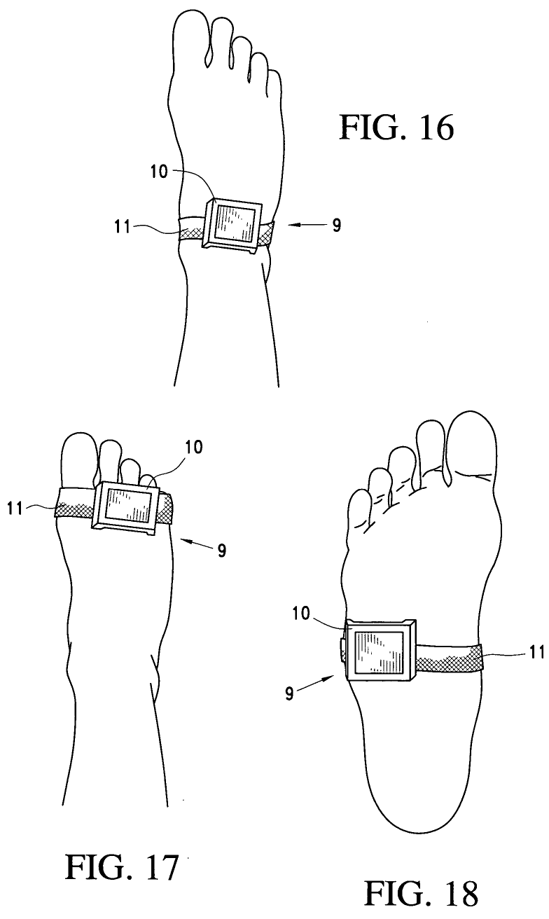

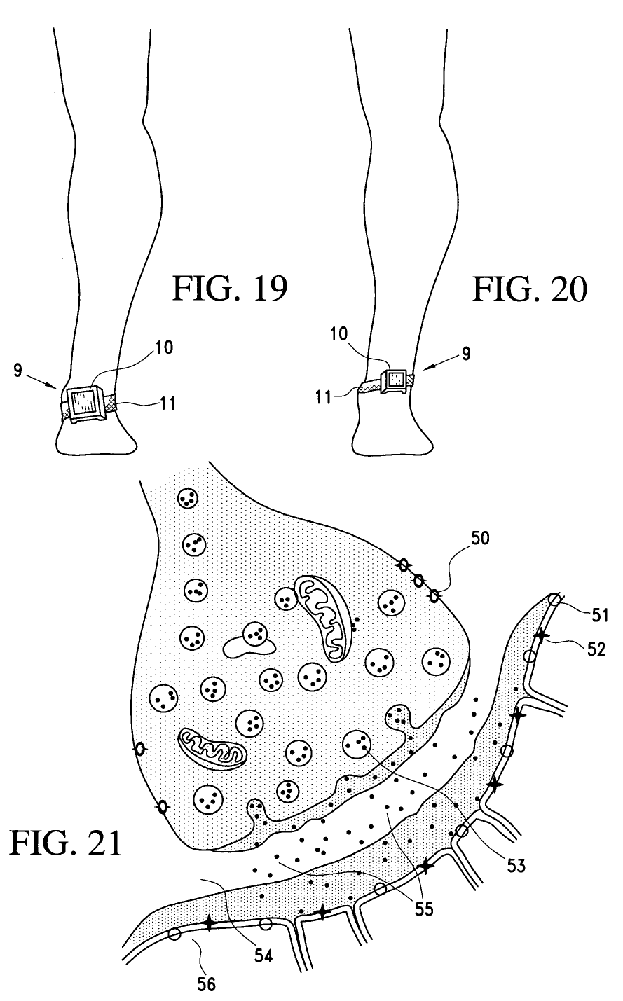

FIG. 15 illustrates the method for applying the wearable SEMF generating device 9 in an exemplary posterior fitment appropriate for treatment of Median Nerve Entrapment (Carpal Tunnel Syndrome) on the posterior of the human right hand, and FIG. 16 shows an exemplary fitment appropriate for treatment of Acute Ankle Sprains on the dorsal, human right foot, in accordance with the present invention. FIG. 17 is a diagram illustrating the method for an exemplary fitment appropriate for treatment of Morton's Neuroma on the dorsal, human right foot, and FIG. 18 shows an exemplary fitment appropriate for treatment of Plantar Fascitis on the plantar, human right foot. FIG. 19 is a diagram illustrating the method for an exemplary fitment appropriate for treatment of Achilles Tendonitis on the posterior, human right ankle, and FIG. 20 shows an exemplary fitment appropriate for treatment of Posterior Tibial Tendonitis on the posterior, human right ankle, in accordance with the present invention.

### INFORMAL STUDIES TO DETERMINE EFFECTIVENESS OF THE INVENTION

Other sections of this application refer to a fifteen or so musculoskeletal disorders that this invention will treat, however at the onset of the invention's development, the inventors were acting on reports from the National Institute for Occupational Safety and Health (NIOSH) regarding the prevalence of Medial Nerve Entrapment, more commonly known as Carpal Tunnel Syndrome (CTS). In America alone there are over 8 million current cases of CTS with an additional 850,000 new cases annually. CTS is painful and crippling to the sufferer. Causes of CTS are varied and include pregnancy, menopause, PMS, rheumatoid arthritis, renal failure, high blood pressure and repetitive movement trauma such as sewing, typing, writing and using hand tools. The medical community, as with many other afflictions, has a typical treatment scenario for CTS, starting with splints and modification of work environments, moving onto anti-inflammatory analgesics and corticosteroids with ultimately more than 50% of cases requiring surgery.

The applicants posited that treating CTS with a Static Electro Magnetic Field would be more effective than a splint, as effective as medication without being invasive and definitely lead to the avoidance of surgery. Thus, the majority of the preliminary studies to determine effectiveness of the invention have been done on the wrist, with some associated elbow pain.

The criteria should be noted for all subjects of all of these studies.

All pain should be due to repetitive movement or other trauma

No degenerative diseases

No anti-inflammatory medication for 12 hours

No pain medication for 12 hours

Study A (Sylvester):

Criteria for evaluating effectiveness of the invention for this study were based on [1] Grip Strength measured three times and averaged, pre- and post-application, using a Hydraulic Hand Dynamometer. [2] Bilateral flexion and extension (Range of Motion) measured three times and averaged, pre- and post-application using a Digital Inclinometer.

Six test subjects had Grip Strength and Range of Motion measured on a painful wrist. They then immediately received a one-time application of the invention on said wrist, lasting from 45 to 60 minutes. Grip Strength and Range of Motion were then immediately re-measured on the affected wrist.

All six test subjects showed an increase in all three of the evaluation criteria. Grip Strength increases ranged from 2% to 35%. Flexion increases ranged from 4% to 12%, and Extension increases ranged from 5% TO 20%.

Study B (Sylvester):

Criteria for evaluating effectiveness of the invention for this study were base on the 11 point JHACO pain scale, with 0 (zero) representing no pain and 10 (ten) representing the worst pain ever experienced.

Thirteen test subjects, 4 males and 9 females, ranging in age from 29 years to 73 years were asked to wear the invention for 5 (five) consecutive days, recording their pain levels immediately prior to application and immediately following application. All subjects experienced a diminishment of pain.

Results are as follows:

Average time worn 1.75 hours (30 minutes-5 hours)

Average prior-application pain scale 4.67 (1.6-9)

Average post-application pain scale 3.07 (0.5-8)

Average percent decrease of pain 33.6% (12%-78%)

Study C (Sylvester):

Criteria for evaluating effectiveness of the invention for this study were base on the 11 point JHACO pain scale, with 0 (zero) representing no pain and 10 (ten) representing the worst pain ever experienced. It should be noted, invention application on all subjects was 30 minutes.

Subject 1

Female—40 years—Constant Epicondylitis of the left elbow due to Repetitive Motion Syndrome.

Application 1

Pre-application pain scale 9

Post-application pain scale 5

Pain decrease 44%

Application 2

Pre-application pain scale 9

Post-application pain scale 2

Pain decrease 78%

Subject 2

Female—36 years—Constant wrist pain due to automobile accident.

Application 1

Pre-application pain scale 8

Post-application pain scale 5

Pain decrease 38%

Application 2

Pre-application pain scale 5

Post-application pain scale 3

Pain decrease 40%

Subject 3

Female—44 years—Constant wrist pain due to parrot bite.

Application 1

Pre-application pain scale 5

Post-application pain scale 0

Pain decrease 100%

Application 2

Pre-application pain scale 3

Post-application pain scale 0

Pain decrease 100%

Subject 4

Female—64 years—Constant wrist pain, cause unknown.

Application 1

Pre-application pain scale 6

Post-application pain scale 4

Pain decrease 33%

Application 2

Pre-application pain scale 7

Post-application pain scale 5

Pain decrease 28%

Subject 5

Female—37 years—Constant wrist pain, cause unknown.

Application 1

Pre-application pain scale 5

Post-application pain scale 1

Pain decrease 80%

Application 2

Pre-application pain scale 5

Post-application pain scale 2

Pain decrease 60%

Subject 6

Male—37 years—Constant wrist and elbow pain due to Repetitive Motion Syndrome.

Application 1

Pre-application pain scale 9

Post-application pain scale 6

Pain decrease 33%

Application 2

Pre-application pain scale 8

Post-application pain scale 5

Pain decrease 37%

Persons of skill in the art will appreciate that the present invention makes available a therapeutic apparatus for reducing inflammation and related pain in a living human body using a Static Electro Magnetic Field, the apparatus comprising a Static Electro Magnetic Field generating unit for generating a Static Electro Magnetic Field that is oriented toward a painful portion of the human body, a housing unit to protect and contain the said Static Electro Magnetic Field generating unit and an affixing unit for affixing the said housing unit to the living human body. In use, the method and apparatus of the present invention provides a means for treating inflammation and related pain is due to Median Nerve Entrapment (Carpal Tunnel Syndrome), Medial Epicondylitis (Golfer's Elbow), DeQuervain's Disease (Blackberry Thumb), Forearm Tendonitis, Wrist Tendonitis, Trigger Finger, Intersection Syndrome, Hand Arm Vibration Syndrome (HAVS), Lateral Epicondylitis (Tennis Elbow), Ulnar Nerve Entrapment (Cubital Tunnel Syndrome), Acute Ankle Sprains, Peroneal Tendonitis, Achilles Tendonitis, Plantar Fascitis, or Morton's Neuroma (Entrapment Neuropathy).

In the illustrative embodiment, Static Electro Magnetic Field generating unit 9 generates a non-oscillating Static Electro Magnetic Field between about 24 Gauss to 26 Gauss at 0 Hertz and includes solenoid induction coil 12, a non-oscillating (or DC) voltage source preferably comprising one or more rechargeable batteries, a single pole single throw switch having an opened and closed state, an operating state indicator such as a light emitting diode and an electrical connector for use as a battery re-charging terminal.

The apparatus 9 preferably includes a solenoid induction coil 12 comprising an electrical conductor such as a copper wire wound around and supported by a non ferrous coil bobbin or form. The non-oscillating voltage source, charging socket 20 and single pole single throw switch 27 are electrically connected or coupled together in a series circuit while the solenoid induction coil 12 and light emitting diode 18 are coupled together in a parallel circuit. The housing unit 10 is a thermoplastic molded case to protect and contain the static electro magnetic field generating device 9 and, preferably, a hook and loop watchband 11 releasably affixes housing unit 10 to the human body.

Having described preferred embodiments of a new and improved method, it is believed that other modifications, variations and changes will be suggested to those skilled in the art in view of the teachings set forth herein. It is therefore to be understood that all such variations, modifications and changes are believed to fall within the scope of the present invention as defined by the appended claims.

What is claimed is:

1. A portable, wearable therapeutic apparatus for reducing inflammation and related pain in a living human body using a Static Electro Magnetic Field, comprising:
a Static Electro Magnetic Field generating circuit for generating a Static Electro Magnetic Field having a selected field strength of that is oriented toward a painful portion of the human body;
said Static Electro Magnetic Field generating circuit including a magnetic flux generating circuit element powered by a non-oscillating portable power supply, said portable power supply being selectively enabled by operation of an enabling switch;
a housing configured to substantially enclose and protect said Static Electro Magnetic Field generating circuit; and
a releasable strap configured to affix said housing unit against the living human body.

2. The portable, wearable therapeutic apparatus of claim 1, wherein said Static Electro Magnetic Field generating unit generates a non-oscillating Static Electro Magnetic Field having a field strength in the range of 24 Gauss to 26 Gauss at 0 Hertz.

3. The portable, wearable therapeutic apparatus of claim 1, wherein said Static Electro Magnetic Field generating unit flux generating circuit element comprises a solenoid induction coil, and
wherein said non-oscillating portable power supply comprises one or more rechargeable batteries.

4. The portable, wearable therapeutic apparatus of claim 3, further comprising a light emitting diode connected with said coil.

5. The portable, wearable therapeutic apparatus of claim 3, further comprising a charging socket configured to permit recharging the portable power supply.

6. The portable, wearable therapeutic apparatus of claim 3, wherein said solenoid induction coil comprises at least one winding of an electrical conductor supported by a non ferrous coil form;
wherein said non-oscillating voltage source, said charging socket and said single pole single throw switch are coupled together in a series circuit; and
wherein said solenoid induction coil and said light emitting diode are connected in parallel.

7. The portable, wearable therapeutic apparatus of claim 3, wherein said housing unit is a molded plastic case contoured along a body contact surface and said strap comprises a web segment or band sized to affix said housing unit to a selected contact surface of the human body, said web segment or band having first and second terminal ends carrying hook and loop fastener segments.

8. A method for treating inflammation or pain in a selected area of the human body with a portable, wearable device, comprising the method steps of:
(a) providing a Static Electro Magnetic Field generating circuit for generating a Static Electro Magnetic Field having a selected field strength, said circuit being adapted for orientation toward the selected area of the body; said Static Electro Magnetic Field generating circuit including a magnetic flux generating circuit element powered by a non-oscillating portable power supply, said portable power supply being selectively enabled by operation of an enabling switch;
(b) providing a housing configured to substantially enclose and protect said Static Electro Magnetic Field generating circuit;
(c) providing a releasable strap configured to affix said housing unit against the body;
(d) identifying the selected area of the body for treatment with a Static Electro Magnetic Field; and
(e) fitting said housing enclosing said Static Electro Magnetic Field generating circuit against or proximate the selected area of the body in an orientation permitting exposure of the painful area to the Static Electro Magnetic Field generated by the Static Electro Magnetic Field generating circuit.
(f) Energizing said Static Electro Magnetic Field generating circuit to expose the selected area of the body to the Static Electro Magnetic Field.

9. The method for treating inflammation or pain in an area of the human body with a portable, wearable device of claim 8, wherein step (e) comprises fitting said housing in an orientation permitting treatment of inflammation and related pain due to Median Nerve Entrapment or Carpal Tunnel Syndrome.

10. The method for treating inflammation or pain in an area of the human body with a portable, wearable device of claim 8, wherein step (e) comprises fitting said housing in an orientation permitting treatment of inflammation and related pain due to Medial Epicondylitis or Golfer's Elbow.

11. The method for treating inflammation or pain in an area of the human body with a portable, wearable device of claim 8, wherein step (e) comprises fitting said housing in an orientation permitting treatment of inflammation and related pain due to DeQuervain's Disease or Blackberry Thumb.

12. The method for treating inflammation or pain in an area of the human body with a portable, wearable device of claim 8, wherein step (e) comprises fitting said housing in an orientation permitting treatment of inflammation and related pain due to Forearm Tendonitis.

13. The method for treating inflammation or pain in an area of the human body with a portable, wearable device of claim 8, wherein step (e) comprises fitting said housing in an orientation permitting treatment of inflammation and related pain due to Wrist Tendonitis.

14. The method for treating inflammation or pain in an area of the human body with a portable, wearable device of claim 8, wherein step (e) comprises fitting said housing in an orientation permitting treatment of inflammation and related pain due to Trigger Finger.

15. The method for treating inflammation or pain in an area of the human body with a portable, wearable device of claim 8, wherein step (e) comprises fitting said housing in an orientation permitting treatment of inflammation and related pain due to Intersection Syndrome.

16. The method for treating inflammation or pain in an area of the human body with a portable, wearable device of claim 8, wherein step (e) comprises fitting said housing in an orientation permitting treatment of inflammation and related pain due to Hand Arm Vibration Syndrome or HAVS.

17. The method for treating inflammation or pain in an area of the human body with a portable, wearable device of claim 8, wherein step (e) comprises fitting said housing in an orientation permitting treatment of inflammation and related pain due to Lateral Epicondylitis or Tennis Elbow.

18. The method for treating inflammation or pain in an area of the human body with a portable, wearable device of claim 8, wherein step (e) comprises fitting said housing in an orientation permitting treatment of inflammation and related pain due to Ulnar Nerve Entrapment or Cubital Tunnel Syndrome.

19. The method for treating inflammation or pain in an area of the human body with a portable, wearable device of claim 8, wherein step (e) comprises fitting said housing in an orientation permitting treatment of inflammation and related pain is due Acute Ankle Sprain.

20. The method for treating inflammation or pain in an area of the human body with a portable, wearable device of claim 8, wherein step (e) comprises fitting said housing in an orientation permitting treatment of inflammation and related pain due to Peroneal Tendonitis.

21. The method for treating inflammation or pain in an area of the human body with a portable, wearable device of claim 8, wherein step (e) comprises fitting said housing in an orientation permitting treatment of inflammation and related pain due to Achilles Tendonitis.

22. The method for treating inflammation or pain in an area of the human body with a portable, wearable device of claim 8, wherein step (e) comprises fitting said housing in an orientation permitting treatment of inflammation and related pain due to Plantar Fascitis.

23. The method for treating inflammation or pain in an area of the human body with a portable, wearable device of claim 8, wherein step (e) comprises fitting said housing in an orientation permitting treatment of inflammation and related pain due to Morton's Neuroma or Entrapment Neuropathy.
